(19) United States
(12) Patent Application Publication
(10) Pub. No.: US 2012/0309798 A1
(43) Pub. Date: Dec. 6, 2012

(54) Tumour Treatment Agents and Method

(75) Inventors: Rolf Larsson, Uppsala (SE); Peter Nygren, Uppsala (SE); Joakim Gullbo, Uppsala (SE); Gunnar Westman, Harryda (SE)

(73) Assignee: Vivolux AB

(21) Appl. No.: 13/516,274

(22) PCT Filed: Dec. 8, 2010
(86) PCT No.: PCT/SE2010/000289

§ 371 Date: Aug. 27, 2012

(30) Foreign Application Priority Data
Dec. 17, 2009 (SE) 0901574-4

(51) Int. Cl. A61K 31/4402; A61P 35/00; C07D 213/53

(52) U.S. Cl. 514/357; 546/331

(57) ABSTRACT

N1-(3-Methoxypropyl)-2-(pyridylmethylidene)-hydrazine-1-carbothioamide (I) and its Cu²⁺, Pd²⁺ and Pt²⁺ complexes are effective anti-tumor agents. Also disclosed are compositions comprising (I) and its Cu²⁺, Pd²⁺ and Pt²⁺ complexes, and the use of the compositions in the treatment of malignant tumors.

### FIELD OF THE INVENTION

The invention relates to agents for treating tumors, pharmaceutical compositions comprising such agents, methods of treating tumors comprising the use of such compositions, and methods of preparing the agents and the compositions. Malignant tumors as understood herein comprise malignant solid tumors defined as an abnormal mass of malignant tissue that usually does not contain cysts or liquid areas, and hematological neoplasms.

### BACKGROUND OF THE INVENTION

It is commonplace that malignant tumors, if not treated in time, will kill the patient. Examples of solid malignant tumors are sarcomas, carcinomas, and lymphomas. Examples of hematological neoplasms are lymphocytic leukemia and myelogenous leukemia.

While a great number of chemical agents, such as cisplatin, cytarabine, melphalan, vinicristine, epoposide and doxorubicin, are useful in the treatment of malignant tumors, there is a great need for more efficient and/or more tumor-specific anti-tumor agents.

### Short Description of the Invention

According to the present invention is disclosed the anti-tumor agent N1-(3-methoxypropyl)-2-(pyridylmethylidene)-hydrazine-1-carbothioamide (I), in the following also identified as VLX50, of the structural formula

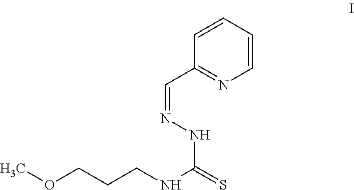

Also disclosed and comprised by the term “compound of the invention” are anti-tumor active metal complexes of hydrazinecarbothioamide I, such as the Cu²⁺ complex II (VLX60) of the structural formula

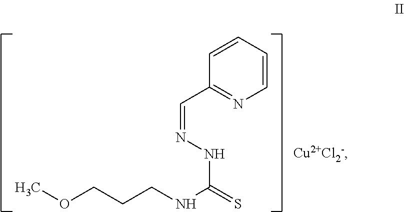

the Pd²⁺ complex III (VLX61) of the structural formula

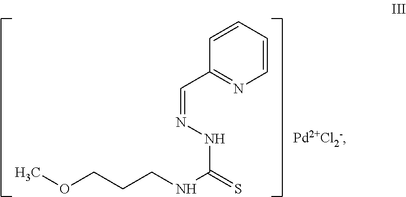

and the Pt²⁺ complex IV (VLX62) of the structural formula

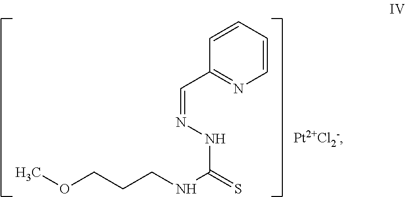

While, in the complexes VLX60, VLX61, and VLX62, the counter-ion for Cu²⁺, Pd²⁺, and Pt²⁺ depicted is chloride, any other suitable counter-ion may be used, such as Br⁻, I⁻, HSO₄⁻, SO₄²⁻, HPO₄²⁻, NO₃⁻, MeSO₃⁻ and is comprised by VLX60, VLX61, VLX62.

According to the invention are also disclosed complexes of hydrazinecarbothioamide I with any of Mn²⁺, Zn²⁺, Co²⁺, Ni²⁺, Bi³⁺, which are of same utility.

According to the invention is also disclosed the use of any of VLX50, VLX60, VLX61, and VLX62 in the treatment of malignant tumors.

According to the invention is also disclosed a pharmaceutical composition comprising any of VLX50 and a suitable pharmaceutical carrier. According to the invention is furthermore disclosed a pharmaceutical composition comprising any of VLX60, VLX61, VLX62 and suitable pharmaceutical carrier. In the composition VLX50, VLX60, VLX61 or VLX62 is present in a therapeutically effective amount.

Solid malignant tumors that can be treated by the anti-tumor agent of the invention include, but are not restricted to: tumors of the bladder, such as squamous cell carcinoma and urothelial carcinomas; bone tumors, such as cartilage tumors and osteogenic tumors; breast tumors, such as breast adenocarcinoma; tumors of the colon, such as colorectal adenocarcinoma; tumors of endocrine glands, such as adrenal cortical carcinoma; tumors of the esophagus, such as adenocarcinoma; gastric tumors, such as adenocarcinoma, carcinoids, primary gastric lymphoma; tumors of the head and the neck, such as squamous cell carcinoma, laryngeal tumors, optic nerve glioma, oral squamous cell carcinoma, retinoblastoma; tumors of the kidney, such as renal cell carcinoma and papillary renal cell carcinoma; tumors of the liver, such as hepatocellular carcinoma, hepatoblastoma, undifferentiated carcinoma; tumors of the lung, such as small cell carcinoma and non-small cell lung cancer; tumors of the nervous system, such as glioma and medulloblastoma; ovarian tumors, such as epithelial tumors; tumors of the skin, such as melanoma; soft tissue tumors, such as angiomyxoma, liposarcoma, malignant melanoma of soft parts; squamous cell cancer; tumors of the testis, such as germ cell tumors; thyroid tumors, such as anaplastic carcinoma and papillary carcinoma; tumors of the uterus, such as carcinoma of the cervix and endometrial carcinoma. Leukemia that can be treated by the anti-tumor agent of the invention includes, but is not restricted to, acute lymphoblastic leukemia; chronic lymphocytic leukemia; acute myelogenous leukemia; chronic myelogenous leukemia; T-cell prolymphocytic leukemia.

The present invention also relates to a pharmaceutical composition comprising a therapeutically effective amount of the compound of the invention and a pharmaceutically acceptable carrier.

The term “pharmaceutically acceptable carrier” as used herein refers to any carrier, diluent, excipient, suspending agent, lubricating agent, adjuvant, vehicle, delivery system, emulsifier, disintegrant, absorbent, preservative, surfactant, colorant, flavorant, or sweetener.

For the treatment of solid tumors, the composition of the invention may be administered orally, parenterally, topically, rectally, vaginally, intraventricularly, or by implantation in a in sustained release dosage form. The term parenteral as used herein includes intraventricular, subcutaneous, intravenous, intramuscular, intraperitoneal, intrathecal, and intracranial injection or infusion. For the treatment of hematological neoplasms, the preferred route of administration is parenteral, in particular intravenous.

When administered parenterally, the composition will normally be in a unit dosage, sterile injectable form (solution, suspension or emulsion) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents, for example, as solutions in 1,2-butanediol. Acceptable vehicles and solvents that may be employed are, for instance, water, saline, Ringer's solution, dextrose solution, isotonic sodium chloride solution, and Hanks' solution. In addition, sterile oils can be employed as solvents or suspending mediums.

Sterile saline is a preferred aqueous carrier. The carrier may contain minor amounts of additives, such as substances that enhance solubility, isotonicity, and chemical stability, e.g., anti-oxidants, buffers and preservatives.

When administered orally, the composition will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like. The composition of the invention can be administered as a capsule or tablet containing a single or divided dose of the compound of the invention. The composition can also be administered as a sterile solution, suspension, or emulsion, in a single or divided dose. Tablets may contain carriers such as lactose and corn starch, and/or lubricating agents such as magnesium stearate. Capsules may contain diluents including lactose and dried corn starch. A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Molded tablets may be made by molding in a suitable machine, a mixture of the powdered active ingredient and a suitable carrier moistened with an inert liquid diluent.

The compound of the invention may also be administered rectally in the form of suppositories. These compositions can be prepared by mixing the drug with a suitable non-irritating excipient which is solid at room temperature, but liquid at rectal temperature, and, therefore, will melt in the rectum to release the drug. Such materials include cocoa butter, beeswax, and polyethylene glycols.

The composition of the invention also may utilize controlled release technology. Thus, for example, the compound of the invention may be incorporated into a hydrophobic polymer matrix for controlled release over a period of days. The composition of the invention may then be molded into a solid implant, or externally applied patch, suitable for providing efficacious concentrations of the compound of the invention over a prolonged period of time without the need for frequent re-dosing. Such controlled release films are well known to the art.

In another embodiment, the carrier is a solid biodegradable polymer or mixture of biodegradable polymers with appropriate time release characteristics and release kinetics. The composition of the invention may then be molded into a solid implant suitable for providing efficacious concentrations of the compounds of the invention over a prolonged period of time without the need for frequent re-dosing. The composition of the present invention can be incorporated into the biodegradable polymer or polymer mixture in any suitable manner known to one of ordinary skill in the art and may form a homogeneous matrix with the biodegradable polymer, or may be encapsulated in some way within the polymer, or may be molded into a solid implant. In one embodiment, the biodegradable polymer or polymer mixture is used to form a soft “depot” containing the pharmaceutical composition of the present invention that can be administered as a flowable liquid, for example, by injection, but which remains sufficiently viscous to maintain the pharmaceutical composition within the localized area around the injection site. The degradation time of the depot so formed can be varied from several days to a few months and even longer, depending upon the polymer selected and its molecular weight. By using a polymer composition in injectable form, even the need to make an incision may be eliminated. In any event, a flexible or flowable delivery “depot” will adjust to the shape of the space it occupies with the body with a minimum of trauma to surrounding tissues. The pharmaceutical composition of the present invention is used in amounts that are therapeutically effective, and may depend upon the desired release profile, the concentration of the pharmaceutical composition required for the anti-tumor effect, and the length of time that the pharmaceutical composition has to be released for treatment.

In an ex vivo phase II trial the anti-tumor agent of the invention exhibited a broad spectrum of activity second only to cisplatin with respect to relative solid tumor activity. Moreover, the CLL/PBMC IC 50 ratio is indicative of a high therapeutic index ex vivo. The ex vivo findings are supported by VLX50 inducing significant in vivo activity in PHTC ovarian cancer cells at low toxicity.

The compound of the invention is capable of depleting intracellular iron in malignant tumors. While not wishing to be bound by theory, the inventors consider intracellular iron depletion to be a potentially important strategy for cancer therapy, in particular in the pharmacological treatment of malignant tumors. The mechanism of anti-tumor action of the agent of the invention was explored by a drug specific gene expression signature to probe the CMAP and GSEA databases. From the CMAP data base strong connections to iron chelators whereas GSEA connected the signature to hypoxia and HIF alfa signaling. These results strongly suggest that VLX50 induces intracellular iron depletion, which subsequently leads to hypoxia signaling through the HIF alfa pathway. This hypothesis is supported by the finding that extracellular iron abolishes the effect of VLX50. Further confirmation was provided by the finding that the anti-tumor agent of the invention efficiently decreases free intracellular iron in tumor cells.

Cancer cells have a higher requirement for iron than normal cells as reflected by increased numbers of transferrin receptors and increased ferritin content in tumor tissues (Richardson et al., 2009). The higher demand for iron may at least partly be explained by the increased activity of iron dependent enzymes such as ribonucleotide reductase, a rate limiting step in DNA synthesis (Shao et al., 2006a). It is known that the activity and expression of RR is increased in tumor cells indicating a high level DNA synthesis in these cells (Elford et al., 1970). Alternatively or additionally, the activation of HIF1 alfa transcription may cause cell death mediated by iron depletion (Ke and Costa, 2006). HIF1alfa is a transcription factors which under conditions of adequate oxygen supply is hydroxylated by the Fe-containing enzyme prolyl hydroxylase leading to proteasome degradation and reduced transcriptional activity (Ke and Costa, 2006). However, under hypoxic conditions or Fe depletion, the enzyme is rendered inactive resulting in increased transcription of HIF1 alfa regulated genes. Notable in this context is the HIF1 alfa dependent increase in expression of the apoptosis-inducing gene BNIP (Chong et al., 2002) and the growth and metastasis suppressor Ndrg-1 (Kovacevic et al., 2008). The potential involvement of these genes is supported by VLX50 significantly increasing their expression. In addition, Fe-depletion may lead to differential expression of a range of cell cycle molecules including cyclin D1-3, p21 and CDK2, which may contribute to the G1/S arrest observed after Fe depletion induced by VLX50 and other iron chelators (Nurtjahja-Tjendraputra et al., 2007; Yu et al., 2007)

Since iron chelators have previously been reported to induce cell death through the inhibition of ribonucleotide reductase this principle could provide a novel therapeutic strategy for cancer. Desferrioxamine is an extracellular iron chelator currently used in the clinic for treatment of iron overload disorders (Richardson et al., 2009). In addition, desferrioxamine has also demonstrated anti-proliferative activity against a wide variety of tumor cells and anticancer activity has been reported in clinical trials (Donfrancesco et al., 1995; Donfrancesco et al., 1990). More recently the intracellular Fe chelator triapine has been developed as a potential anticancer agent with an excellent preclinical activity in many tumor models and is currently undergoing Phase I and II clinical trials (Chaston et al., 2003; Finch et al., 2000; Richardson et al., 2009). The semithiocarbazone triapine has been shown to be a potent inhibitor of ribeonucleotide reductase (Finch et al., 2000). However, in addition to ribeonucleotide reductase inhibition, triapine has been reported to be redox active leading to ROS formation (Shao et al., 2006b) potentially adding to the reported drug induced toxicity. ROS generation could lead to several toxicological consequences as a result of oxidative injury to important biomolecules such as DNA, proteins and lipids (Kalinowski and Richardson, 2007). In contrast to triapine, the semithiocarbazone VLX50 does not appear to induce ROS formation, a distinct advantage in a clinical setting.

Thus, according to the present invention, is disclosed a method of treating a malignant tumor in a patient by administration of a therapeutically effective intracellular iron-depleting amount of VLX50 but also of VLX60, VLX61, and VLX62.

The term “therapeutically effective amount” of the compound of the invention means an amount effective, when administered to a human or non-human patient, to provide a therapeutic benefit, such as decelerating or stopping the growth of a solid tumor or to make the tumor shrink or vanish or, in respect of a hematological neoplasm, to stabilize or substantially reduce the number of malignant blood cells in the circulation or to even eradicate them. A therapeutically effective amount may be one of from 0.01 mg/kg to 100 mg/kg and even more. For a given kind of tumor, the therapeutically effective amount depends on the mode of administration, systemic administration to be effective generally requiring a higher amount than administration at the tumor site.

The term “treating” a malignant tumor refers to inhibiting or decelerating the growth of the tumor or causing regression of the tumor or preventing the tumor from spreading.

### ABBREVIATIONS

ALL Acute Lymphocytic Leukemia
AML Acute Myelocytic Leukemia
CLL Chronic Lymphocytic Leukemia
CML Chronic Myelocytic Leukemia
NHL Non-Hodgkins Lymphoma
PHTC Primary cultures of Human Tumor Cells from patients
IC50 50% Inhibitory Concentration
WBC White Blood Cells
RBC Red Blood Cells
ROS Reactive oxygen species

### DESCRIPTION OF THE FIGURES

FIG. 1a is a representative photomicrograph of PHTC with ovarian carcinoma, stained with May-Grunwald-Giemsa;

FIG. 1b is a diagram illustrating the effect of VLX50 on different PHTC from patients with ovarian carcinoma (n=14) and on the normal epithelial cell line RPE hTERT (n=3) The results are presented as survival index and expressed as mean values+SEM;

FIGS. 2a-2c are staple diagrams illustrating the pharmacological activity of VLX50 in respect of:
a) Ex vivo response rate in a panel of PHTC representing a range of diagnoses;
b) Solid/hematologic tumor ratio (S/H ratio) for VLX50 and six prior art anti-cancer agents (n=98);
c) PBMC (n=4)/CLL (n=9) IC50 ratio for VLX50 and the six prior art anti-cancer agents;

FIG. 3a is a group of three staple diagrams illustrating the in vivo activity of VLX50 in hollow fiber cultures of PHTC from two patients with ovarian carcinoma (OC1; OC2) and the cell line CCRF-CEM (CEM). The results are presented as net growth and expressed as mean values+SEM (n=8);

FIG. 3b is a diagram illustrating the effect of VLX50 and of vehicle (control) on the weight of NMRI male mice;

FIG. 3c is a table illustrating the effect of VLX50 on WBC, RBC, Hb and platelet count;

FIG. 4a is a diagram illustrating the concentration dependent VLX50-induced growth Inhibition determined by phase contrast time-lapse microscopy; hourly confluence analyses were carried out during culturing of MCF-7 tumor cells in 24 well plates;

FIGS. 4b-4d are staple diagrams showing the effect of VLX50 on average cell density measured by Arrayscan II (4b), DNA fragmentation (4c) and caspase-3/7 activity (4d) at 24 h-72 h from start. The results are expressed as % of the untreated control and presented as mean values+SEM for three experiments;

FIGS. 5a-5d illustrate the effect of VLX50 on tumor cell survival in a panel of ten cell lines representative of various forms of drug resistance: Diagram showing concentration/response curves for the cell lines (FIG. 5a); diagram showing cell line delta values for each cell line defined as log IC50 minus the mean of the log IC50 for all ten cell lines. Deflections to the right and left indicate lower and higher sensitivity, respectively (FIG. 5b); Table listing delta value means for all ten cell lines in respect of five selected standard anti-cancer agents (FIG. 5c); Table listing calculated resistance factors for five resistance mechanisms. The resistance factor is defined as IC50 in the resistant cell line/IC50 in the parental cell line (FIG. 5d);

FIG. 6a is a staple diagram illustrating the effect of extracellular Fe on VLX50 induced cell death;

FIG. 6b is a diagram illustrating the effect of VLX50 on intracellular Fe concentration measured by a fluorescent probe.

### DESCRIPTION OF PREFERRED EMBODIMENTS

### Materials and Methods

Methyl hydrazinecarbodithioate. Potassium hydroxide (13.2 g, 0.2 mol) is dissolved in 15 ml water and 12 ml 2-propanol. The solution was cooled to 5° C. Hydrazine hydrate (10 g, 0.2 mol) was added slowly under stirring. Carbon disulfide (15.2 g, 0.2 mol) was added drop-wise, and the solution stirred for 120 min at 5° C. Methyl iodide (28.3 g, 0.2 mol) was added slowly. After the addition stirring was continued for 2 hrs. The precipitate was filtered off and dried. Yield 6.8 g, 37%. M.p. 81-83° C. ¹H NMR (CDCl₃): 2.4 ppm (3H, s), 4.0 ppm (2H, broad), 8.7 ppm (1H, broad).

Methyl 2-(2-pyridylmethylene)-hydrazinecarbodithioate. Methyl hydrazinecarbodithioate (2.2 g, 18.3 mmol) was dissolved in 2-propanol (10 ml). After adding 2-pyridine aldehyde (2.0 g, 18.6 mmol) drop-wise to the solution stirring was continued for 90 min. The reaction mixture was stored in a refrigerator overnight, and the precipitate filtered off and dried. Yield 2.2 g, 55%. M.p. 171-173° C. ¹H NMR (DMSO-d₆): 2.6 ppm (3H, s), 7.4 ppm (1H dd), 7.9 ppm (1+1H dd, d), 8.2 ppm (1H, s), 8.6 ppm (1H, dd).

N1-(3-Methoxypropyl)-2-(pyridylmethylidene)-hydrazine-1-carbothioamide (VLX50). Methyl 2-(2-pyridinylmethylene)-hydrazinecarbodithioate (0.5 g, 2.4 mmol) and 1-amino-3-methoxypropane (0.25 g, 2.8 mmol) was dissolved in dry methanol and refluxed for 12 hrs. The precipitate was filtered off and recrystallized from ethyl acetate. Yield 0.18 g, 30%. M.p. 108-110° C. ¹H NMR (CDCl₃): 2.0 ppm (2H, tt), 3.4 ppm (3H, s), 3.6 ppm (2H, t), 3.8 ppm (2H, t) 7.3 ppm (1H, dd), 7.7 ppm (1H, t), 7.8 ppm (1H, s), 7.9 ppm (1H, d), 8.2 ppm (1H, broad singlett) 8.6 ppm (1H, d), 9.0 ppm (1H, s). VLX50 is a known compound commercially obtainable from Maybridge plc (Fischer Scientific).

Cu²⁺ complex of N1-(3-methoxypropyl)-2-(pyridylmethylidene)-hydrazine-1-carbothioamide, (VLX60). To a solution of VLX50 (41 mg) in 5 ml of ethanol is added 60 mg of CuCl₂ in 2 ml of ethanol. The mixture is stirred for 3 hrs at room temperature. The green precipitate is filtered off and washed with ethanol. Yield 74 mg. ¹H NMR (DMSO-d₆): could not be recorded due to Cu²⁺ being paramagnetic.

Pd²⁺ complex of N1-(3-methoxypropyl)-2-(pyridylmethylidene)-hydrazine-1-carbothioamide, (VLX61). To a solution of VLX50 (41 mg) in 5 ml of ethanol is added 42 mg of PdCl₂ in 37 ml of ethanol. The mixture is stirred for 20 hrs at room temperature. The yellow precipitate is filtered off and washed with ethanol. Yield 35 mg. ¹H NMR (DMSO-d₆): 1.8 ppm (2H, t), 3.4 ppm (3H, s), 3.5 ppm (4H, t), 8.0 ppm (1H, s), 8.3 ppm (1H, t), 8.5 ppm (1H, d).

Pt²⁺ complex of N1-(3-methoxypropyl)-2-(pyridylmethylidene)-hydrazine-1-carbothioamide, (VLX62). To a solution of VLX50 (41 mg) in 0.5 ml of ethanol is added 42 mg of PdCl₂ in 0.5 ml of ethanol. The mixture is stirred for 20 hrs at reflux temperature. The red-brown precipitate is filtered off and washed with ethanol. Yield 65 mg. ¹H NMR (DMSO-d₆): 1.8 ppm (2H, t), 3.6 ppm (7H, broad), 7.8 ppm (2H, m), 8.5 ppm (1H, s), 8.8 ppm (1H, d).

Pharmaceutical compositions. The following illustrate representative pharmaceutical dosage forms containing the compound of the invention, for therapeutic use in humans.

(a) Tablet. Compound of formula I (2.0 mg), lactose (76.0 mg), povidone (14.0 mg) croscarmellose sodium (12.0), microcrystalline cellulose 90.0, magnesium stearate (3.0 mg).
(b) Tablet. Compound of formula II, III or IV (1.0 mg), microcrystalline cellulose (400 mg), starch (50.0 mg), sodium starch glycolate (14.0), magnesium stearate (5.0 mg).
(c) Hard gelatin capsule. Compound of formula I (10.0 mg), colloidal silicon dioxide (1.5 mg), lactose 430 mg, pregelatinized starch (120 mg), magnesium stearate (3.0 mg).
(d) Solution for Injection. Compound of formula I (5.0 mg), sodium dihydrogen phosphate (10.0 mg), disodium hydrogen phosphate (5.7 mg), sodium chloride (4.5 mg), 01.0 N sodium hydroxide solution q.s. (pH adjustment to 7.0-7.5), water for injection q.s. ad 1 mL.
(e) Solution for Injection. Compound of formula II, III or IV (0.5 mg/ml), sodium dihydrogen phosphate (1.3 mg), disodium hydrogen phosphate (0.6 mg), polyethylene glycol 400 (200.0 mg), 0.1 N sodium hydroxide solution q.s. (pH adjustment to 7.0-7.5), water for injection q.s. ad 1 mL.

Cell culture. Patient tumor samples (98) and preparations (4) of normal peripheral blood mononuclear cells (PBMC), detailed in Table 1, were used to determine the activity of VLX50 and, for comparison, six other cytotoxic drugs chosen to represent different mechanistic classes.

TABLE 1
Median IC50 and range for different diagnoses in response to VLX50.

| Diagnosis | Median IC50 (uM) | Range | n |
|---|---|---|---|
| ALL | 0.63 | 0.055-11.21 | 21 |
| AML | 5.35 | 0.67-40 | 10 |
| CLL | 1.01 | 0.035-9.40 | 9 |
| CML | 5.13 | 2.10-6.42 | 3 |
| NHL | 2.33 | 0.28-40 | 13 |
| Breast cancer | 8.23 | 0.87-40 | 7 |
| Ovarian carcinoma | 3.46 | 0.26-40 | 14 |
| Lung cancer | 13.60 | 0.51-40 | 6 |
| Colon cancer | 40 | 0.49-40 | 6 |
| Renal cancer | 40 | 23.84-40 | 7 |
| Assorted*) | 7.80 | 1.60-13.27 | 2 |
| PBMC | 8.92 | 2.88-13.27 | 4 |

*)Assorted tumors: one appendix cancer and one pseudomyxomo peritonei.

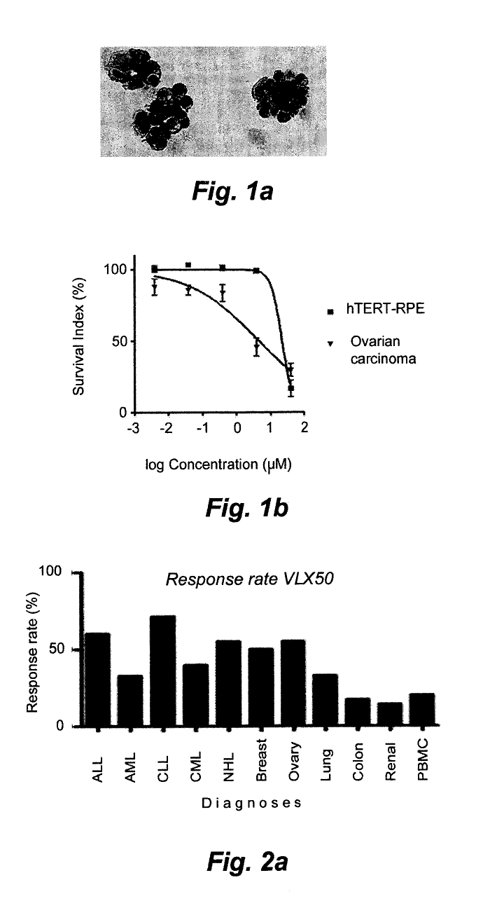

The tumor samples were obtained by bone marrow/peripheral blood sampling, routine surgery or diagnostic biopsy. Leukemic cells and PBMCs were isolated by 1.077 g ml-1 Ficoll-Paque centrifugation (Larsson et al., 1992). Tumor tissue from solid tumor samples was minced into small pieces and tumor cells were isolated by collagenase dispersion followed by Percoll density gradient centrifugation (Csoka et al., 1994). The sampling of primary tumor cells was approved by the local ethics committee at Uppsala University Hospital. Cell viability was determined by trypan blue exclusion test and the proportion of tumor cells in the preparation was judged by inspection of May-Grunvald-Giemsa stained cytospin slides (FIG. 1a). All samples used in this study contained more than 70% tumor cells.

The cell lines used in this study were breast cancer MCF7 and hTERT-RPE (normal epithelial cell line) obtained from American Type Culture Collection (ATCC) and Clontech (Palo Alto, Calif.), respectively. The remaining cell line panel used has been described in detail previously (Dhar et al., 1996) and consists of the parental cell lines RPM; 8226 (myeloma), CCRF-CEM (leukemia), NCI-H69 (small cell lung cancer), U-937 GTB (lymphoma), ACHN (renal cell carcinoma) and the drug-resistant sub-lines 8226/Dox40, 8226/LR5, CEM/VM-1, U-937 VCR, and H69AR. The sub-line 8226/Dox40 was exposed to 0.24 μg/ml doxorubicin once a month and over-expresses Pgp/MDR1/ABCB1 (Dalton et al., 1986). The 8226/LR5 sub-line was exposed to 1.53 μg/ml of melphalan at each change of medium; resistance is suggested to be associated with increased levels of glutathione as well as genes involved in cell cycle and DNA-repair (Mulcahy et al., 1994). U937 VCR was continuously cultured in the presence of 10 ng/ml vincristine and the resistance is proposed to be tubulin associated (Botling et al., 1994). H69AR was alternately fed with drug-free medium and medium containing 0.46 doxorubicin and over-expresses MRP1/ABCC1 Cole (Cole et al., 1992). CEM/VM-1 was cultured in drug-free medium and could be grown for 3-4 months without loss of resistance against teniposide which is proposed to be topoisomerase II associated (Bugg et al., 1991; Danks et al., 1988). The resistant phenotypes were stable for more than three months. Normal epithelial hTERT-RPE cells were cultured in in modified Eagles medium nutrient mixture F-12 Ham. The hTERT-RPE cell culture were supplemented with 10% heat-inactivated fetal calf serum, 2 mM glutamine, 100 μg/ml streptomycin and 100 U/ml penicillin (all from Sigma Aldrich Co, St Louis, Mo.) at 37° C. in humidified air containing 5% CO₂. The remaining cell lines cells were grown in culture medium RPMI-1640 supplemented with 10% heat-inactivated fetal calf serum, 2 mM glutamine, 100 μg/ml streptomycin and 100 U/ml penicillin (Sigma) at the same conditions. The resistant cell lines were tested regularly for maintained resistance to the selected drugs. Growth and morphology of all cell lines were monitored on a weekly basis.

Fluorometric Microculture Cytotoxicity Assay. The Fluorometric Microculture Cytotoxicity Assay, FMCA, described in detail previously (Lindhagen et al., 2008), is based on measurement of fluorescence generated from hydrolysis of fluorescein diacetate (FDA) to fluorescein by cells with intact plasma membranes. Cells were seeded in the drug-prepared 384-well plates using the pipetting robot Precision 2000 (Bio-Tek Instruments Inc., Winooski, Vt.). The number of cells per well were 2500-5000. Two columns without drugs served as controls and one column with medium only served as blank. The plates were incubated for 72 h and then transferred to an integrated HTS SAIGAN Core System consisting of an ORCA robot (Beckman Coulter) with CO2 incubator (Cytomat 2C, Kendro, Sollentuna, Sweden), dispensor module (Multidrop 384, Titertek, Huntsville, Ala.), washer module (ELx 405, Bio-Tek Instruments Inc), delidding station, plate hotels, barcode reader (Beckman Coulter), liquid handler (Biomek 2000, Beckman Coulter) and a multipurpose reader (FLUOstar Optima, BMG Labtech GmbH, Offenburg, Germany) for automated FMCA. Quality criteria for a successful assay included a mean coefficient of variation of less than 30% in the control and a fluorescence signal in control wells of more than 5 times the blank.

Multiparametric high content screening assays. To study the cell death characteristics a multi-parametric high content screening (HCS) assay was used (Cellomics cytotoxicity HitKit™) and an HCS assay for measurement of apoptosis, which has been described in detail previously (Lovborg et al., 2004). The cells (1500 cells/well) were seeded into flat-bottomed 96-well plates (Perkin Elmer Inc., Wellesley, Mass.) and were left to attach before addition of drugs. For the cytotoxicity assay the cytotoxicity HitKit™ reagents (Cellomics Inc., Pittsburgh, Pa., USA) was used according to the manufacturer's instructions. Multi-parameter cytotoxicity HitKit™ contains a nuclear dye, a cell permeability dye, and a lysosomal mass/pH indicator. In the apoptosis assay FAM-DEVD-FMK (part of the CaspaTag Kit, Chemicon, Temecula, Calif.) at a final concentration of 20 μM was added one hour before the end of the drug exposure to stain activated caspase-3 and partly caspase-7. The staining solution was removed and the plates were washed twice with PBS followed by a 30 min fixation in 3.7% formaldehyde and nuclear staining with 10 μM Hoechst 33342 (Sigma). Plates were then washed twice. The plates were centrifuged before each aspiration to avoid loss of cells detached due to toxic stimuli. Processed plates were kept at +4° C. for up to 24 h before analysis. Plates were analyzed using the ArrayScan™ HCS software (Cellomics Inc). The system is a computerized automated fluorescence-imaging microscope that automatically identifies stained cells and reports the intensity and distribution of fluorescence in individual cells. Images were acquired for each fluorescence channel, using suitable filters with 20× objective. In each well at least 800 cells were analyzed. Images and data were stored in a Microsoft SQL database.

Phase contrast microscopy. Time lapse phase contrast microscopy was performed using an automated Incucyte phase contrast microscope. MCF-7 cells (10,000/well) were plated on 24-well ImageLock plates (Essen Instruments, Ann Arbor, Mich.) and cultured in RPMI 1640 media containing 10% fetal bovine serum and antibiotics. The plates were immediately placed into IncuCyte imaging system (Essen Instruments). The chamber is designed to fit into a standard, humidified, CO₂ incubator in an atmosphere of 5% CO₂, and a moving objective allows the cell culture to be stationary while images are captured at different positions from well to well. Images were collected at hourly intervals starting 30 minutes after addition of the plate to the IncuCyte-FLR chamber. Drug treatment was performed 24 hours after the plates were placed in the Incucyte. Cell density was calculated using the Incucyte software. Movies were generated using IncuCyte software (Essen Instruments) at three frames/second, which is equivalent to 30 minutes of culture/second.

Measurement of intracellular iron. The fluorescent membrane permeable Fe sensor, Phen Green (Invitrogen AB, Göteborg, Sweden) was used to measure changes in intracellular free iron concentration. Fluorescence of the PhenGreen indicators is quenched upon binding Fe²⁺ and Fe³⁺. The emission intensity of the PhenGreen FL indicator depends on both the metal ion's concentration and the indicator's concentration. MCF-7 cells were harvested and diluted to 500,000 per mL in complete medium, loaded with 10 μM PhenGreen diacetate for 45 minutes and plated to flat-bottomed 96W microtiter plates. The plates were washed ×2 with PBS, resuspended in 180 μl PBS per well and subsequently analyzed by a plate reading fluorometer Fluostar Optima which is programmed to scan fluorescence once a minute for 20 minutes with a break after 5 minutes, during which 20 μl PBS, or VLX50 (100 μM or 500 μM) was added.

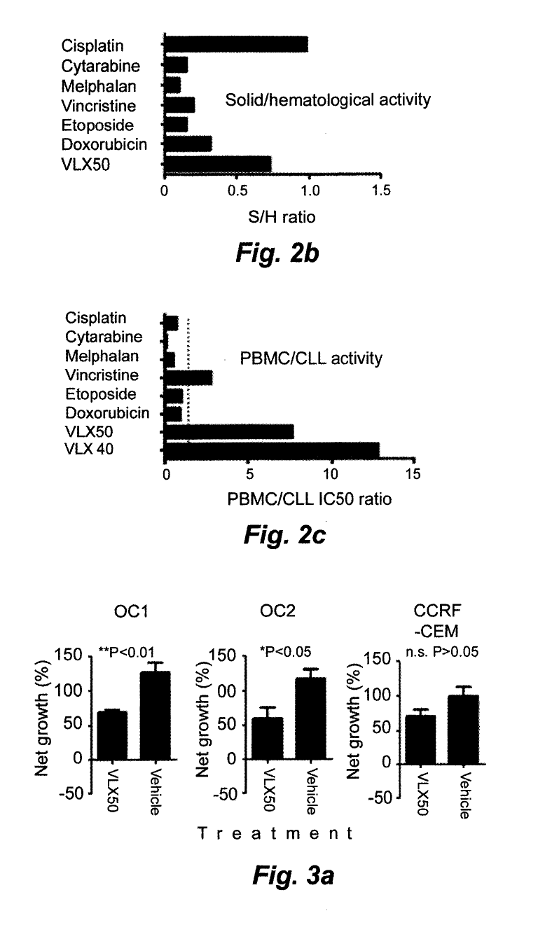

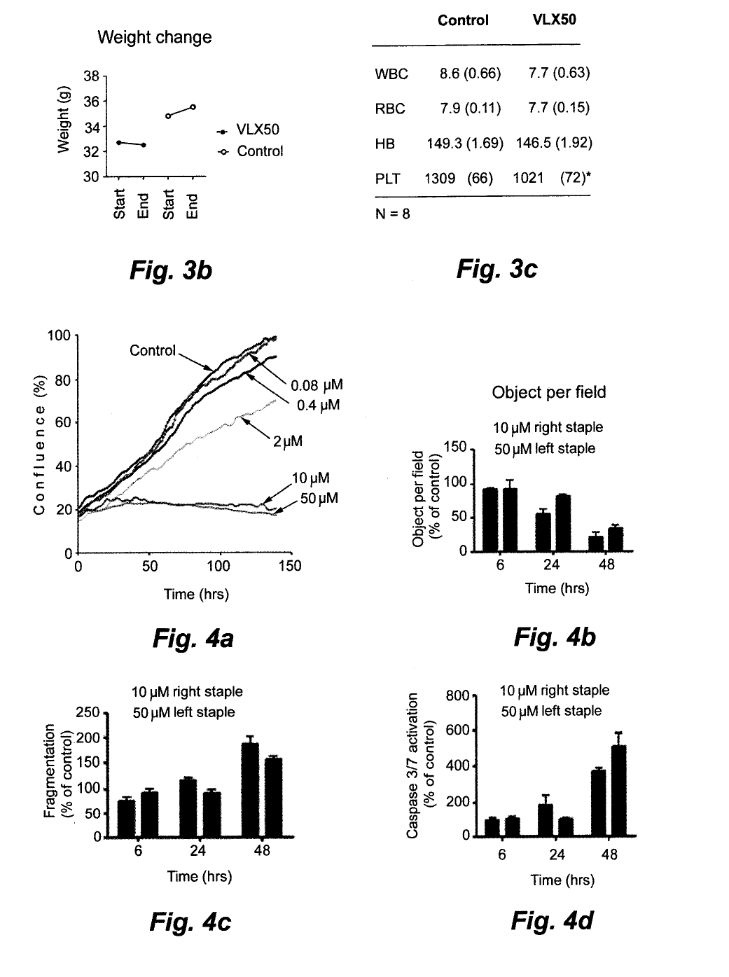

In vivo studies. Cells from two ovarian carcinoma patients and the cell line CEM were cultured inside semi-permeable polyvinylidene fluoride fibers and assessed by the hollow fiber assay (Friberg et al., 2005; Jonsson et al., 2000). The fibers were implanted subcutaneously into NMRI male mice (Scanbur, Sollentuna Sweden), which were treated with a single dose (0.76 mg/mouse) of VLX50 subcutaneously, or vehicle only (n=animals/group). Fibers were retrieved after 6 days and cell density evaluated (FIG. 3a) using the MTT (3-[4,5-dimethylthiazol-2-yl]-2,5-diphenyltetrazolium bromide)-assay (Alley et al. 1988). The method is based on the conversion of MTT to blue formazan crystals by living cells. The formazan was extracted by DMSO as previously described (Jonsson et al. 2000), and optical density (OD) read at 570 nm. Cell density for each fiber on retrieval day was expressed as net growth, defined as (OD retrieval day—OD implantation day)/OD implantation day, i.e. the percent change in cell density in the fibers during the 6 days of in vivo experiment. The animals were observed regarding behavior and weight gain throughout the experiment. Blood samples (200 μl) were obtained through the orbital plexus after anesthetization with isofluran just before euthanasia, and analyzed for hematological parameters. Four animals were caged per cage and fed a commercial diet (Lactamin AB, Sweden), water being given ad libitum.

Data analysis and statistics. Small Laboratory Information and Management System (Kelley et al., 2004) was used for screening data management and analysis. Raw fluorescence data files were loaded into the SLIMS software which calculates percent inhibition according to the formula: Percent inhibition=100×(x-negative control/positive control-negative control) −1, where x denotes fluorescence from experimental wells. SLIMS also identifies and corrects systematic spatial errors. More than or equal to 50% mean inhibition in Ovca PHTC was set as the criteria for qualifying as hit compound. Structural similarity to other compounds in the library was calculated based on a structural fingerprint consisting of binary vectors representing structures located within the compound and which are automatically computed for each compound loaded into the program. The Z′-value was calculated to evaluate the quality and usefulness of the assay in the screening setting using the equation: Z′=1−[(3SDposcontrol+3SDnegcontrol)/(Meanposcontrol−Meannegcontrol)] where SD and mean are the standard deviation and mean values of screening raw data from wells with untreated cells (positive control) and blank wells (negative control), respectively (Zhang et al., 1999).

Dose-response data were analyzed using calculated survival index values and the software program GraphPadPrism4 (GraphPad Software Inc., San Diego, Calif., USA). Data was processed using non-linear regression to a standard sigmoidal dose-response model to obtain IC50-values (inhibitory concentration 50%).

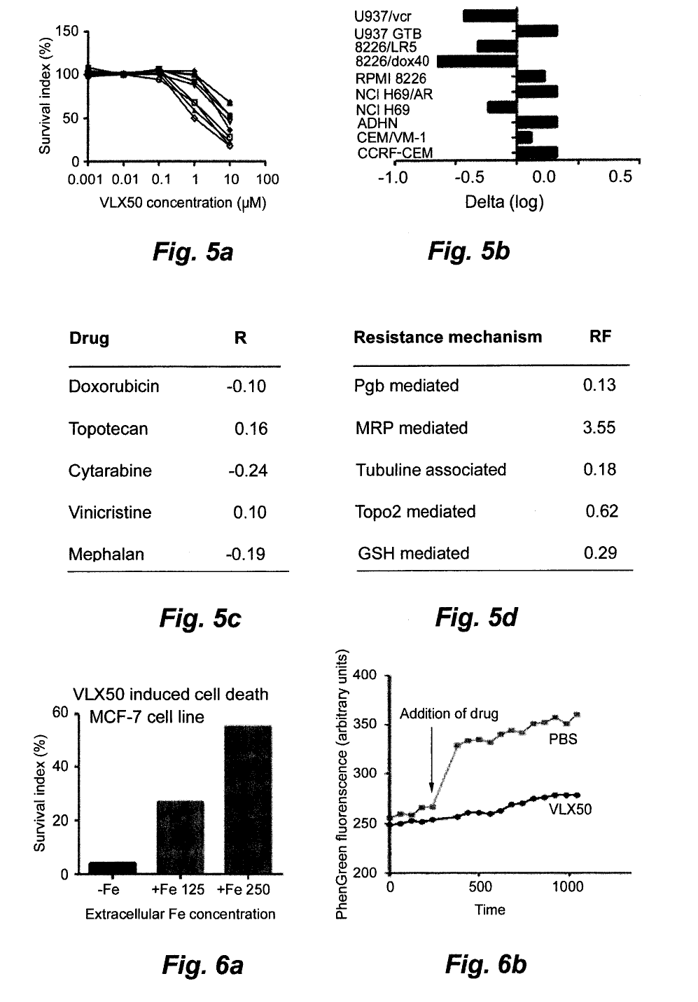

Response rate was defined as the fraction of samples having a survival index below the median at the concentration from the dose-response curves showing the largest standard deviation. For VLX50 this concentration was 4 μM (cf, FIGS. 3b, 5a). The relative effect of a drug on solid and hematological tumors was indicated by the S/H ratio, defined as the ratio between the total response rates for the solid and the hematological samples (cf, FIG. 2b).

Diagnosis-specific activity ex vivo. To examine the effect of VLX50 in a setting close to the clinic, its anti-tumor activity was studied in 98 tumor samples from patients with a variety of solid and hematological cancer diagnoses as well as in four PBMC. The IC50-values ranged from diagnoses with a median IC₅₀ of below 5 μM such as CLL, ALL, ovarian cancer and lymphoma to the more resistant colon and renal cancer samples with IC50 above 40 μM (Table 1).

Cancers of the breast, lung, CML, AML and PBMC displayed intermediate sensitivity to VLX50. In FIG. 2a the response rates for VLX50 at 4 μM for the patient samples are listed according to diagnoses. Corroborating the IC50 patterns the lymphocytic malignancies showed the highest response rates followed by breast and ovarian cancer whereas PBMC, colon and renal cancer had the lowest response rates. Lung cancer, AML and CML had intermediate response rates. The relative effect of VLX50 and six standard cytotoxic drugs, in solid and hematological tumor samples, expressed as the S/H ratio, is shown in FIG. 2b. VLX50 had a ratio of 0.73 indicating a relatively high activity against solid tumors, second only to cisplatin (S/H ratio of 1.2). The remaining drugs had a S/H ratio below 0.5. The results for the standard drugs are consistent with their main clinical use. To roughly estimate tumor cell specificity, drug effect in cells from CLL and normal PBMC were compared (FIG. 2c), demonstrating a significantly higher activity against the malignant phenotype with a PBMC/CLL median IC50 ratio of 7.6. Of the tested standard cytotoxic drugs only vincristine were significantly more active in CLL than in PBMC. Notably, both cytarabin and melphalan showed significantly higher activity in PBMC than in CLL cells (t-test, p<0.05). No difference was observed for doxorubicin, etoposide and cisplatin.

The effect of VLX50 on tumor cell survival in a panel of ten cell lines is shown in FIG. 5a.

In vivo activity in PHTC cultures of ovarian carcinoma. Activity in vivo was determined in hollow fiber cultures of PHTC from ovarian carcinoma patients subcutaneously implanted in mice (FIG. 3a; n=8 each in test and control groups). After a single dose of 760 μg/mouse significant growth inhibition compared to vehicle treatment were observed in the two PHTC cultures (P<0.05 and P<0.01, respectively). The difference between the VLX50 treated group and the control group did not reach statistical significance in the control cell line CCRF-CEM (P>0.05). VLX50 induced a small but significant reduction in weight gain compared to the control group (P<0.05; FIG. 3b). A significant decrease in platelet counts was also evident (P<0.05). There was no difference in WBC, RBC, and hemoglobin values (FIG. 3c).

Pharmacological profiling in a resistance based cell line panel. When comparing the log IC50 patterns with some commonly used cytotoxic agents, VLX50 showed low correlation (R=−0.24−0.19) indicating absence of cross resistance to these standard drugs. In response VLX50, an increased sensitivity compared with parental cell lines was observed in the sub-lines with Pgp-tubulin-GSH and topo II-mediated drug resistance with resistance factors ranging from 0.13-0.62, thus indicating collateral sensitivity. For NCl H69 and its resistant sub-line CEM/VM-1 the resistance factor was 3.55 suggesting the involvement of MRP in mediating VLX50 resistance (FIG. 5d). The resistance factors for some tested standard agents (FIG. 5d) confirmed the expected resistance pattern of the drug resistant sub-lines (not shown).

Mode of VLX50 induced cell death. VLX50 was profiled with respect to mode of action using time lapse phase contrast microscopy and multi-parameter analysis using Arrayscan II. The effect of VLX50 on growth and viability was delayed with little or no effect observable at 24 h (FIGS. 4a and 4b). At 48-72 h there was a gradual decrease in cell density and a parallel increase in caspase-3 activity and DNA fragmentation (FIG. 4c). Phase contrast images of the cells at this time point revealed a typical apoptotic morphology with condensed nuclei surrounded by a bright halo (FIG. 4a). The increase in DNA fragmentation and caspase activation preceded the increase in cell membrane permeability which is compatible with classical apoptosis.

Mechanism of action. Mechanistic exploration was performed using gene expression analysis of drug treated tumor cell cultures to generate a drug specific signature. The breast cancer cell line MCF-7 was treated with VLX50 or vehicle (DMSO) and analyzed for gene expression using the Affymetrix U1300plus chip. A drug specific query signature was generated based on 100 most up-regulated genes. This query signature was subsequently submitted to the GSEA and the Connectivity map databases and strong connections to hypoxia inducible factor (HIF1 alfa) and iron chelators were retrieved. The mechanistic hypothesis of VLX50 causing intracellular iron depletion subsequently leading to hypoxia signaling was first tested by adding extracellular iron to VLX treated MCF-7 cell cultures which resulted in a dose-dependent decrease in VLX50 activity (FIG. 6a). The mechanism was confirmed by direct measurements of drug induced decrease in intracellular iron concentration (FIG. 6b).

### REFERENCES

Alley, M. C. et al. (1988). Feasibility of drug screening with panels of human tumor cell lines using a microculture tetrazolium assay. Cancer Res 48, 589-601.
Bugg, B. Y. et al. (1991). Expression of a mutant DNA topoisomerase II in CCRF-CEM human leukemic cells selected for resistance to teniposide. Proc Natl Acad Sci USA 88, 7654-7658.
Chaston, T. B. et al. (2003). Examination of the antiproliferative activity of iron chelators: multiple cellular targets and the different mechanism of action of triapine compared with desferrioxamine and the potent pyridoxal isonicotinoyl hydrazone analogue 311. Clin Cancer Res 9, 402-414.
Chong, T. W. et al. (2002). A mycobacterial iron chelator, desferri-exochelin, induces hypoxia-inducible factors 1 and 2, NIP3, and vascular endothelial growth factor in cancer cell lines. Cancer Res 62, 6924-6927.
Cole, S. P. et al. (1992). Overexpression of a transporter gene in a multidrug-resistant human lung cancer cell line. Science 258, 1650-1654.
Csoka, K. et al. (1994). Cytotoxic drug sensitivity testing of tumor cells from patients with ovarian carcinoma using the fluorometric microculture cytotoxicity assay (FMCA). Gynecol Oncol 54, 163-170.
Csóka, K. et al. (1997). In vitro determination of cytotoxic drug response in ovarian carcinoma using the fluorometric microculture cytotoxicity assay (FMCA). Int J Cancer 72, 1008-1012.
Dalton, W. S. et al. (1986). Characterization of a new drug-resistant human myeloma cell line that expresses P-glycoprotein. Cancer Res 46, 5125-5130.
Danks, M. K. et al. (1988). Altered catalytic activity of and DNA cleavage by DNA topoisomerase II from human leukemic cells selected for resistance to VM-26. Biochemistry 27, 8861-8869.
Dhar, S. et al. (1996). Anti-cancer drug characterisation using a human cell line panel representing defined types of drug resistance. Br J Cancer 74, 888-896.
Donfrancesco, A. et al. (1995). Deferoxamine followed by cyclophosphamide, etoposide, carboplatin, thiotepa, induction regimen in advanced neuroblastoma: preliminary results. Italian Neuroblastoma Cooperative Group. EurJ Cancer 31A, 612-615.
Donfrancesco, A. (1990). Effects of a single course of deferoxamine in neuroblastoma patients. Cancer Res 50, 4929-4930.
Elford, H. L. et al. (1970). Ribonucleotide reductase and cell proliferation. I. Variations of ribonucleotide reductase activity with tumor growth rate in a series of rat hepatomas. J Biol Chem 245, 5228-5233.
Finch, R. A. et al. (2000). Triapine (3-aminopyridine-2-carboxaldehyde-thiosemicarbazone): A potent inhibitor of ribonucleotide reductase activity with broad spectrum antitumor activity. Biochem Pharmacol 59, 983-991.
Friberg, L. E. et al. (2005). Pharmacokinetic-pharmacodynamic modelling of the schedule-dependent effect of the anti-cancer agent CHS 828 in a rat hollow fibre model. European journal of pharmaceutical sciences: official journal of the European Federation for Pharmaceutical Sciences 25, 163-173.
Jonsson, E. et al. (2000). Determination of drug effect on tumour cells, host animal toxicity and drug pharmacokinetics in a hollow-fibre model in rats. Cancer Chemother Pharmacol 46, 493-500.
Kalinowski, D. S, and Richardson, D. R. (2007). Future of toxicology-iron chelators and differing modes of action and toxicity: the changing face of iron chelation therapy. Chem Res Toxicol 20, 715-720.
Ke, Q. and Costa, M. (2006). Hypoxia-inducible factor-1 (HIF-1). Mol Pharmacol 70, 1469-1480.
Kelley, B. P. et al. (2004). A flexible data analysis tool for chemical genetic screens. Chem Biol 11, 1495-1503.
Kovacevic, Z et al. (2008). The iron-regulated metastasis suppressor, Ndrg-1: identification of novel molecular targets. Biochim Biophys Acta 1783, 1981-1992.
Larsson, R. (1992). Laboratory determination of chemotherapeutic drug resistance in tumor cells from patients with leukemia, using a fluorometric microculture cytotoxicity assay (FMCA). Int J Cancer 50, 177-185.
Lövborg, H. et al. (2004). Multiparametric evaluation of apoptosis: effects of standard cytotoxic agents and the cyanoguanidine CHS 828. Mol Cancer Ther 3, 521-526.
Mulcahy, R. T. et al. (1994). Up-regulation of gamma-glutamylcysteine synthetase activity in melphalan-resistant human multiple myeloma cells expressing increased glutathione levels. Cancer Chemother Pharmacol 34, 67-71.
Nurtjahja-Tjendraputra, E. et al. (2007). Iron chelation regulates cyclin D1 expression via the proteasome: a link to iron deficiency-mediated growth suppression. Blood 109, 4045-4054.
Richardson, D. R. et al. (2009). Cancer cell iron metabolism and the development of potent iron chelators as anti-tumour agents. Biochim Biophys Acta 1790, 702-717.
Shao, J. et al (2006a). Ribonucleotide reductase inhibitors and future drug design. Curr Cancer Drug Targets 6, 409-431.
Shao, J. et al. (2006b). A Ferrous-Triapine complex mediates formation of reactive oxygen species that inactivate human ribonucleotide reductase. Mol Cancer Ther 5, 586-592.
Subramanian, A. et al. (2005). Gene set enrichment analysis: a knowledge-based approach for interpreting genome-wide expression profiles. Proc Natl Acad Sci USA 102, 15545-15550.
Wickström, M. et al. (2007). Pharmacological profiling of disulfuram using human tumor cell lines and human tumor cells from patients. Biochem Pharmacol 73, 25-33.
Yu, Y. et al. (2007). Tuning cell cycle regulation with an iron key. Cell Cycle 6, 1982-1994.
Zhang, J. H. et al. (1999). A Simple Statistical Parameter for Use in Evaluation and Validation of High Throughput Screening Assays. Journal of biomolecular screening: the official journal of the Society for Biomolecular Screening 4, 67-73.

1. (canceled)

2. A compound of formula II

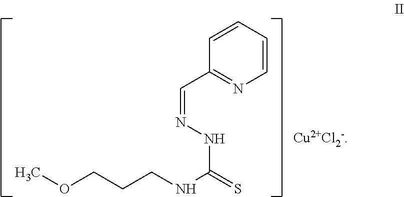

3. A compound of formula III

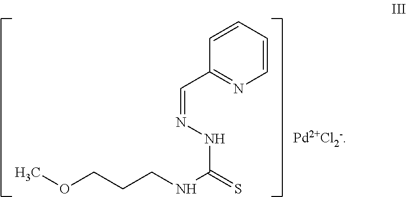

4. A compound of formula IV

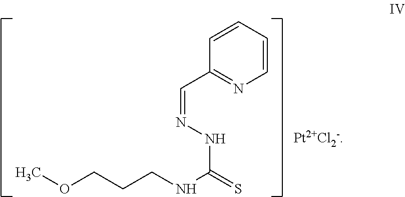

5. The compound of claim 2, wherein Cl₂⁻ is substituted by any of Br⁻, I⁻, HSO₄⁻, SO₄²⁻, HPO₄²⁻, NO₃⁻, or MeSO₃⁻.

6. (canceled)

7. A pharmaceutical composition comprising a therapeutically effective amount of any of compounds I to IV, wherein the amount is effective in the treatment of a malignant tumor, and a pharmaceutically acceptable carrier:

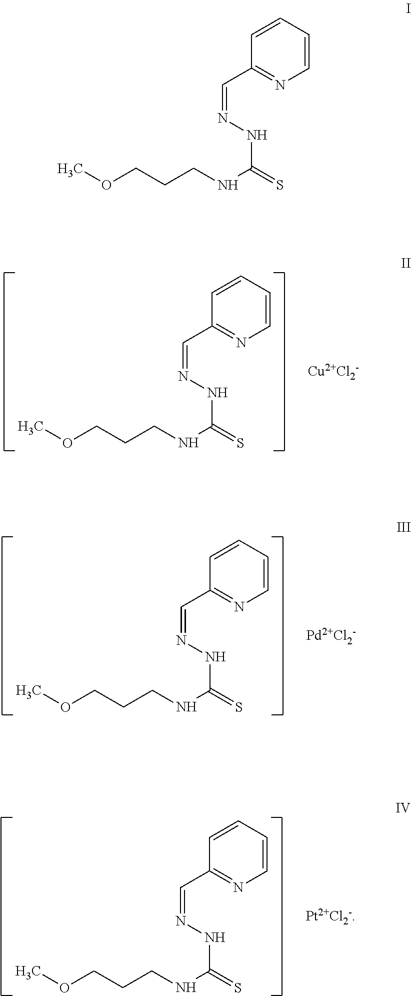

8. The pharmaceutical composition of claim 7, adapted for injection or infusion.

9. The pharmaceutical composition of claim 7, adapted for per-oral administration.

10. A method of treating a malignant tumor in a patient, comprising administering to the patient the pharmaceutical composition of claim 7.

11. The compound of claim 3, wherein Cl₂⁻ is substituted by any of Br⁻, I⁻, HSO₄⁻, SO₄²⁻, HPO₄²⁻, NO₃⁻, or MeSO₃⁻.

12. The compound of claim 4, wherein Cl₂⁻ is substituted by any of Br⁻, I⁻, HSO₄⁻, SO₄²⁻, HPO₂²⁻, NO₃⁻, or MeSO₃⁻.

13. The pharmaceutical composition of claim 7, comprising a therapeutically effective amount of compound I.

14. The pharmaceutical composition of claim 7, comprising a therapeutically effective amount of compound II.

15. The pharmaceutical composition of claim 7, comprising a therapeutically effective amount of compound III.

16. The pharmaceutical composition of claim 7, comprising a therapeutically effective amount of compound IV.

17. A method of treating a malignant tumor in a patient, comprising administering to the patient the pharmaceutical composition of claim 8.

18. A method of treating a malignant tumor in a patient, comprising administering to the patient the pharmaceutical composition of claim 9.

19. A method of treating a malignant tumor in a patient, comprising administering to the patient the pharmaceutical composition of claim 13.

20. A method of treating a malignant tumor in a patient, comprising administering to the patient the pharmaceutical composition of claim 14.

21. A method of treating a malignant tumor in a patient, comprising administering to the patient the pharmaceutical composition of claim 15.

22. A method of treating a malignant tumor in a patient, comprising administering to the patient the pharmaceutical composition of claim 16.
